(19) United States
(12) Patent Application Publication
(10) Pub. No.: US 2005/0261529 A1
(43) Pub. Date: Nov. 24, 2005

(54) Enhancement modifiers for gas hydrate inhibitors

(75) Inventors: Daniel L. Crosby, Sugar Land, TX (US); Gordon T. Rivers, Houston, TX (US); Lynn M. Frostman, Sugar Land, TX (US)

Correspondence Address: MADAN, MOSSMAN & SRIRAM, P.C., 2603 AUGUSTA, SUITE 700, HOUSTON, TX, 77057, US

(73) Assignee: Baker Hughes Incorporated

(21) Appl. No.: 11/129,799

(22) Filed: May 16, 2005

Related U.S. Application Data
(60) Provisional application No. 60/572,022, filed on May 18, 2004.

(51) Int. Cl. C07C 9/00

(52) U.S. Cl. 585/15

(57) ABSTRACT

A method for inhibiting formation of hydrocarbon hydrates in mixtures of water and a hydrate-forming guest molecule involves adding an ion pair to the mixtures in an amount effective to inhibit formation of the hydrocarbon hydrates under conditions otherwise effective to form the hydrocarbon hydrates in the absence of the ion pair. In one non-limiting embodiment of the invention the ion pair includes a cationic component that may be a quaternary ammonium compound or an onium compound and a non-cationic counter-ion component that could be an anionic compound, a non-ionic compound and/or an amphoteric compound. Two specific, suitable non-cationic counter-ion components include sodium dodecyl sulfate and ammonium alkyl ether sulfate.

### CROSS-REFERENCE TO RELATED APPLICATION

This application claims the benefit of U.S. provisional application No. 60/572,022 filed May 18, 2004.

### FIELD OF THE INVENTION

The invention relates to methods and compositions for inhibiting the formation of hydrocarbon hydrates, and most particularly relates, in one non-limiting embodiment, to methods and compositions for inhibiting the formation of hydrocarbon hydrates during the production of oil and gas.

### BACKGROUND OF THE INVENTION

A number of hydrocarbons, especially lower-boiling light hydrocarbons, in formation fluids or natural gas are known to form hydrates in conjunction with the water present in the system under a variety of conditions—particularly at a combination of lower temperature and higher pressure. The hydrates usually exist in solid forms that are essentially insoluble in the fluid itself. As a result, any solids in a formation or natural gas fluid are at least a nuisance for production, handling and transport of these fluids. It is not uncommon for hydrate solids (or crystals) to cause plugging and/or blockage of pipelines or transfer lines or other conduits, valves and/or safety devices and/or other equipment, resulting in shutdown, loss of production and risk of explosion or unintended release of hydrocarbons into the environment either on-land or off-shore. Accordingly, hydrocarbon hydrates have been of substantial interest as well as concern to many industries, particularly the petroleum and natural gas industries.

Hydrocarbon hydrates are clathrates, and are also referred to as inclusion compounds. Clathrates are cage structures formed between a host molecule and a guest molecule. A hydrocarbon hydrate generally is composed of crystals formed by water host molecules surrounding the hydrocarbon guest molecules. The smaller or lower-boiling hydrocarbon molecules, particularly C₁ (methane) to C₄ hydrocarbons and their mixtures, are more problematic because it is believed that their hydrate or clathrate crystals are easier to form. For instance, it is possible for ethane to form hydrates at as high as 4° C. at a pressure of about 1 MPa. If the pressure is about 3 MPa, ethane hydrates can form at as high a temperature as 14° C. Even certain non-hydrocarbons such as carbon dioxide, nitrogen and hydrogen sulfide are known to form hydrates under the proper conditions.

There are two broad techniques to overcome or control the hydrocarbon hydrate problems, namely thermodynamic and kinetic. For the thermodynamic approach, there are a number of reported or attempted methods, including water removal, increasing temperature, decreasing pressure, addition of “antifreeze” to the fluid and/or a combination of these. The kinetic approach generally attempts (a) to prevent the smaller hydrocarbon hydrate crystals from agglomerating into larger ones (known in the industry as an anti-agglomerate and abbreviated AA) and/or; (b) to inhibit and/or retard initial hydrocarbon hydrate crystal nucleation; and/or crystal growth (known in the industry as a kinetic hydrate inhibitor and abbreviated KHI). Thermodynamic and kinetic hydrate control methods may be used in conjunction.

Kinetic efforts to control hydrates have included use of different materials as inhibitors. For instance, onium compounds with at least four carbon substituents are used to inhibit the plugging of conduits by gas hydrates. Additives such as polymers with lactam rings have also been employed to control clathrate hydrates in fluid systems. These kinetic inhibitors are commonly labeled Low Dosage Hydrate Inhibitors (LDHI) in the art. KHIs and even LDHIs are relatively expensive materials, and it is always advantageous to determine ways of lowering the usage levels of these hydrate inhibitors while maintaining effective hydrate inhibition.

Thus, it is desirable if new gas hydrate inhibitors or modifiers for existing hydrate inhibitors were discovered which would yield comparable or improved results over known gas hydrate inhibitors.

### SUMMARY OF THE INVENTION

An object of the invention is to provide a method for inhibiting gas hydrate formation in mixtures of hydrate-forming guest molecules and water where hydrates would otherwise form to a greater extent in absence of the method.

Another object of the invention is to provide gas hydrate inhibitor compositions and/or hydrate inhibitor synergists that are readily produced. These compositions may be blended with other oil field chemistries such as, but not limited to, corrosion, paraffin, scale and/or asphaltene inhibitors.

Still another object of the invention is to reduce the dosage levels of the more expensive components of the gas hydrate inhibitors.

In carrying out these and other objects of the invention, there is provided, in one form, a method for inhibiting formation of hydrocarbon hydrates in a mixture containing water and hydrate-forming guest molecules. The method involves contacting the mixture with an amount of an ion pair effective to inhibit formation of hydrocarbon hydrates. The ion pair includes a first component that can be a cationic low dosage hydrate inhibitor (LDHI), an anionic LDHI, an amphoteric LDHI or a non-ionic LDHI. The ion pair also includes a second counter-ion component. If the first component is a cationic LDHI, the second counter-ion component is either an anionic compound, a non-ionic compound or an amphoteric compound. If the first component is an anionic LDHI, then the second counter-ion component is either a non-ionic compound, an amphoteric compound or a cationic compound. If the first component is an amphoteric LDHI or a non-ionic LDHI, then the second counter-ion component can be either an anionic compound, a cationic compound, a non-ionic compound or an amphoteric compound.

In another non-limiting embodiment of the invention, there is provided a method for inhibiting formation of hydrocarbon hydrates in a mixture containing water and hydrate-forming guest molecules. The method involves contacting the mixture with an amount of an ion pair effective to inhibit formation of hydrocarbon hydrates. The ion pair includes a cationic quaternary onium compound, and a non-cationic counter-ion component that is either an anionic compound, a non-ionic compound or an amphoteric compound.

In another aspect, the invention includes hydrocarbon mixtures inhibited against hydrate formation formed by the methods described above.

### DETAILED DESCRIPTION OF THE INVENTION

In the present invention there are included methods and compositions used therein for inhibiting, retarding, mitigating, reducing, controlling and/or delaying formation of hydrocarbon hydrates or agglomerates of hydrates. The method may be applied to prevent or reduce or mitigate plugging of conduits, pipes, transfer lines, valves, and other places or equipment where hydrocarbon hydrate solids may form under conditions conducive to their formation or agglomeration. The ion pairs of this invention may be active as an anti-agglomerate (AA) and/or as a kinetic inhibitor (KHI), and the invention should be understood as not restricted to one particular mechanism or the other.

The term “inhibiting” is used herein in a broad and general sense to mean any improvement in preventing, controlling, delaying, reducing or mitigating the formation, growth and/or agglomeration of hydrocarbon hydrates, particularly light hydrocarbon gas hydrates in any manner, including, but not limited to kinetically, thermodynamically, by dissolution, by breaking up, by anti-agglomeration other mechanisms, or any combination thereof. Although the term “inhibiting” is not intended to be restricted to the complete cessation of gas hydrate formation, it may include the possibility that formation of any gas hydrate is entirely prevented.

The terms “formation” or “forming” relating to hydrates are used herein in a broad and general manner to include, but are not limited to, any formation of hydrate solids from water and hydrocarbon(s) or hydrocarbon and non-hydrocarbon gas(es), growth of hydrate solids, agglomeration of hydrates, accumulation of hydrates on surfaces, any deterioration of hydrate solids plugging or other problems in a system and combinations thereof.

The present method is useful for inhibiting hydrate formation for many hydrocarbons and hydrocarbon and/or non-hydrocarbon mixtures. The method is particularly useful for lighter or low-boiling, C₁-C₅, hydrocarbon gases, non-hydrocarbon gases or gas mixtures at ambient conditions. Examples of such gases include, but are not necessarily limited to, methane, ethane, ethylene, acetylene, propane, propylene, methylacetylene, n-butane, isobutane, 1-butene, trans-2-butene, cis-2-butene, isobutene, butene mixtures, isopentane, pentenes, natural gas, carbon dioxide, hydrogen sulfide, nitrogen, oxygen, argon, krypton, xenon, and mixtures thereof. These molecules are also termed hydrate-forming guest molecules herein. Other examples include various natural gas mixtures that are present in many gas and/or oil formations and natural gas liquids (NGL). The hydrates of all of these low-boiling hydrocarbons are also referred to as gas hydrates. The hydrocarbons may also comprise other compounds including, but not limited to CO, CO₂, COS, hydrogen, hydrogen sulfide (H₂S), and other compounds commonly found in gas/oil formations or processing plants, either naturally occurring or used in recovering/processing hydrocarbons from the formation or both, and mixtures thereof.

Suitable LDHIs for use in the methods of this invention include, but are not necessarily limited to, ammonium or onium compounds with at least four carbon substituents, including but not necessarily limited to, lactam rings, amides having at least 3 carbon atoms, imides having at least 3 carbon atoms, and halide quaternary amines; and combinations thereof.

In the present invention, substances useful for improving, modifying, extending and/or enhancing the performance of gas hydrate inhibitors are made by adding the appropriate counter-ion. The resulting ion pair is as effective as, if not more effective than, the original gas hydrate inhibitor. In some cases, the amount of original gas hydrate inhibitor used can be reduced by almost half, yet give the same hydrate-inhibiting effect together with the counter-ion. This pairing of ions has sufficient impact on the cost of the gas hydrate inhibitor product and may prove to increase the environmental friendliness of the inhibitor. In one non-limiting theory of the invention, having relatively large low dosage hydrate inhibitor (LDHI) and relatively large counter-ions paired therewith give pairs with increased steric bulk that aids in hydrate inhibition. In an alternate, non-restrictive theory, it is also possible that the counter ion impacts the partitioning (presumably at the liquid interface) of the active molecule between the brine and liquid hydrocarbon phase, when such a liquid hydrocarbon phase is present. This may better position the active molecule to interact with forming hydrate crystals.

It will be appreciated that the counter-ion component is also called a modifier herein, and may also be properly termed an inhibitor synergist when an effect is achieved that is over and above a simple additive effect of the two components.

The scope of the invention includes any appropriate counter-ion to the active LDHI. More specifically, the invention includes anionic, non-ionic and amphoteric counter-ions for a cationic LDHI; a non-ionic, amphoteric and cationic counter-ion for an anionic LDHI, and an anionic non-ionic, cationic or amphoteric counter ion for an amphoteric or non-ionic LDHI. The appropriate counter-ion may or may not display gas hydrate inhibiting behavior independently or on its own. It will further be appreciated that the two counter ions of the ion pairs of this invention may demonstrate no appreciable AA or KHI activity by themselves, or in some non-restrictive embodiments may individually demonstrate KHI behavior but no AA activity, whereas the ion pair combined form an AA or an improved KHI.

In a more specific, non-limiting embodiment of the invention, a suitable ion pair is one where the LDHI is a cationic component that is a quaternary ammonium compound or an onium compound. The non-cationic counter-ion component for this LDHI may be an anionic compound, a non-ionic compound and/or an amphoteric compound.

Suitable onium compounds for use in the composition for the present invention are defined to have a general structure of the following formula A having a cation with a center atom X and an anion Y⁻: 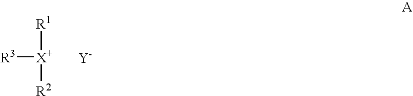
wherein
R¹ and R² each are independently selected from normal or branched alkyls containing a chain of at least 4 carbon atoms, with or without one or more substituents, or one or more heteroatoms;
R³ is an organic moiety containing a chain of at least 4 carbon atoms, with or without one or more substituents, or one or more heteroatoms;
X is S, N—R⁴ or P—R⁴;
R⁴, if present, is selected from H or an alkyl, aryl, alkylaryl, alkenylaryl or alkenyl group, preferably those having from about 1 to about 20 carbon atoms, with or without one or more substituents, or one or more heteroatoms; and
Y⁻ is selected from the group consisting of hydroxide ion (OH⁻), halide ions such as Br⁻ and Cl⁻, carboxylate ions, such as benzoate (C₆H₅COO⁻), sulfate ion (SO₄⁼), organic sulfonate ions, such as 4-toluene sulfonate and CH₃SO₃⁻, and the like and mixtures thereof.

Heteroatoms are defined herein as oxygen, nitrogen, sulfur and phosphorus. When the heteroatom is O, N, or S, suitable substituents or moieties include, but are not necessarily limited to, hydroxyl, ether, carboxylic ester, ketone, amine, amide, nitro, mercaptan, thiol. thioether, sulfide, sulfoxide, sulfone, sulfonic acid, or ether sulfate groups. R¹, R², R³ and R⁴ may contain these groups in a linear or branched manner. When a group is on R^{x} in a branched manner, the group may be referred to as a substituent on R^{x}. When a group is in R^{x} in a linear manner, the group may be referred to as a moiety of R^{x}. When the heteroatom is P, suitable substituents or moieties include, but are not necessarily limited to, phosphonic acid, a phosphonic acid ester or a phosphoric acid ester.

Ammonium and phosphonium compounds of the above formula may also be bound through R⁴ to become pendant groups of a number of oxygen-containing polymers. Suitable oxygen-containing polymers include, but are not limited to polyacrylic acid, polymethacrylic acid, copolymers of acrylic and methacrylic acids, and polymers or co-polymers of poly-N-vinyl-2-pyrrolidone.

Alkyl ammonium and alkyl phosphonium compounds are preferred onium compounds for the composition of the present invention when R⁴ is H or any alkyl or alkenyl group. In these preferred onium compounds, R³ can be optionally selected from the group consisting of —(CH₂CHR⁵—O—)ₙH and —(CH₂CH₂NH—)ₘH, wherein R⁵ is H or methyl; n is an integer from about 5 to about 50; and m is an integer from 1 to about 5. Ammonium and phosphonium compounds are quaternary onium compounds.

Examples of preferred cation moiety for the onium compounds include, but are not limited to, tetrapentylammonium, tripentylbutylammonium, triisopentylbutylammonium, tripentyldecylammonium, triisopentylammonium, tributyloctadecylammonium, tetrabutylphosphonium, tributyl(9-octadecenyl) phosphonium ions and mixtures thereof.

In accordance with formula A, examples of onium compounds include, but are not limited to, tributyldecylammonium, tributylundecylammonium, tributyldodecylammonium, tributyltridecylammonium, tributyltetradecylammonium, tributylpentadecylammonium, tributylhexadecylammonium, tributylheptadecylammonium, tributyloctadecylammonium, tributylnonadecylammonium, tripentyldecylammonium, tripentylundecylammonium, tripentyldodecylammonium, tripentyltridecylammonium, tripentyltetradecylammonium, tripentylpentadecylammonium, tripentylhexadecylammonium, tripentylheptadecylammonium, tripentyloctadecylammonium, tripentylnonadecylammonium, propyldibutyldecylammonium, propyldibutylundecylammo-nium, propyldibutyldodecylammonium, propyldibutyltridecylammonium, propyldibutyltetradecylammonium, propyldibutylpentadecylammonium, propyldibutylhexadecylammonium, propyldibutylheptadecylammonium, propyldibutyloctadecylammonium, propyldibutylnonadecylammonium, allyldibutyldecylammonium, allyldibutylundecylammonium, allyldibutyldodecylammonium, allyldibutyltridecylammonium, allyldibutyltetradecylammonium, allyldibutylpentadecylammonium, allyldibutylhexadecylammonium, allyldibutylheptadecylammonium, allyldibutyloctadecylammonium, allyldibutylnonadecylammonium, methallyldibutyldecylammonium, methallyldibutylundecylammonium, methallyldibutyldodecylammonium, methallyldibutyltridecylammonium, methallyldibutyltetradecylammonium, methallyldibutylpentadecylammonium, methallyldibutylhexadecylammonium, methallyldibutylheptadecylammonium, methallyldibutyloctadecylammonium, methallyldibutylnonadecylammonium, dibutyldidecylammonium, dibutyldiundecylammonium, dibutyldidodecylammonium, dibutylditridecylammonium, dibutylditetradecylammonium, dibutyldipentadecylammonium, dibutyldihexadecylammonium, dibutyldiheptadecylammonium, dibutyldioctadecylammonium and dibutyldinonadecylammonium salts, and mixtures thereof.

Additional preferred “onium” compounds include the phosphonium compounds corresponding to above ammonium compounds. These “onium” compounds include, but are not limited to tributyldecylphosphonium, tributylundecylphosphonium, tributyldodecylphosphonium, tributyltridecylphosphonium, tributyltetradecylphosphonium, tributylpentadecylphosphonium, tributylhexadecylphosphonium, tributylheptadecylphosphonium, tributyloctadecylphosphonium, tributylnonadecylphosphonium, tripentyldecylphosphonium, tripentylundecylphosphonium, tripentyldodecylphosphonium, tripentyltridecylphosphonium, tripentyltetradecylphosphonium, tripentylpentadecylphosphonium, tripentylhexadecylphosphonium, tripentylheptadecylphosphonium, tripentyloctadecylphosphonium, tripentylnonadecylphosphonium, propyldibutyldecylphosphonium, propyldibutylundecylphosphonium, propyldibutyldodecylphosphonium, propyldibutyltridecylphosphonium, propyldibutyltetradecylphosphonium, propyldibutylpentadecylphosphonium, propyldibutylhexadecylphosphonium, propyldibutylheptadecylphosphonium, propyldibutyloctadecylphosphonium, propyldibutylnonadecylphosphonium, allyldibutyldecylphosphonium, allyldibutylundecylphosphonium, allyldibutyldodecylphosphonium, allyldibutyltridecylphosphonium, allyldibutyltetradecylphosphonium, allyldibutylpentadecylphosphonium, allyldibutyhexadecylphosphonium, allyldibutylheptadecylphosphonium, allyldibutyloctadecylphosphonium, allyldibutylnonadecylphosphonium, methallyldibutyldecylphosphonium, methallyldibutylundecylphosphonium, methallyldibutyldodecylphosphonium, methallyldibutyltridecylphosphonium, methallyldibutyltetradecylphosphonium, methallyldibutylpentadecylphosphonium, methallyldibutylhexadecylphosphonium, methallyldibutylheptadecylphosphonium, methallyldibutyloctadecylphosphonium, methallyldibutylnonadecylphosphonium, dibutyldidecylphosphonium, dibutyldiundecylphosphonium, dibutyldidodecylphosphonium, dibutylditridecylphosphonium, dibutylditetradecylphosphonium, dibutyldipentadecylphosphonium, dibutyldihexadecylphosphonium, dibutyldiheptadecylphosphonium, dibutyldioctadecylphosphonium and dibutyldinonadecylphosphonium salts and mixtures thereof.

Also preferred for the present invention are onium compounds wherein zero to five of the CH₂ groups in the longest chains of the onium compound are replaced with one or more of the following groups CHCH₃, CHOH, O, C═O. Thus the onium compound may contain methyl groups, hydroxyl groups, ether groups or linkages, ester groups or linkages, and/or ketone groups. One advantage of such materials is that oxygen atoms in the chains, when present, can improve the biodegradability of the onium compounds. Also, two adjacent CH₂ groups in the longest chains of the onium compound may be replaced with a CH═CH group such that the onium compound may contain one or more carbon to carbon double bonds. The “onium” compounds are named after the parent hydrocarbon and the replacement group(s) in the longest chain are then stated. Thus
CH₃CH₂CH₂CH₂CH₂CH₂CH₂OCH₂CH₂CH₂CH₂N(CH₂CH₂CH₂CH₃)₃
is referred to as tributyldodecylammonium where C5 is replaced with O.

Examples of onium compounds where CH₂ groups in the longest chains are replaced with CHCH₃, CHOH, O, C═O, or CH═CH groups include but are not limited to:

tributyldecylammonium, tributylundecylammonium, tributyldodecylammonium, tributyltridecylammonium, tributyltetradecylammonium, tributylpentadecylammonium, tributylhexadecylammonium, tributylheptadecylammonium, tributyloctadecylammonium, tributylnonadecylammonium, tripentyldecylammonium, tripentylundecylammonium, tripentyldodecylammonium, tripentyltridecylammonium, tripentyltetradecylammonium, tripentylpentadecylammonium, tripentylhexadecylammonium, tripentylheptadecylammonium, tripentyloctadecylammonium, tripentylnonadecylammonium, propyldibutyldecylammonium, propyldibutylundecylammonium, propyldibutyldodecylammonium, propyldibutyltridecylammonium, propyldibutyltetradecylammonium, propyldibutylpentadecylammonium, propyldibutylhexadecylammonium, propyldibutylheptadecylammonium, propyldibutyloctadecylammonium, propyldibutylnonadecylammonium, allyldibutyldecylammonium, allyldibutylundecylammonium, allyldibutyldodecylammonium, allyldibutyltridecylammonium, allyldibutyltetradecylammonium, allyldibutylpentadecylammonium, allyldibutylhexadecylammonium, allyldibutylheptadecylammonium, allyldibutyloctadecylammonium, allyldibutylnonadecylammonium, methallyldibutyldecylammonium, methallyldibutylundecylammonium, methallyldibutyldodecylammonium, methallyldibutyltridecylammonium, methallyldibutyltetradecylammonium, methallyldibutylpentadecylammonium, methallyldibutylhexadecylammonium, methallyldibutylheptadecylammonium, methallyldibutyloctadecylammonium, methaltyldibutylnonadecylammonium, dibutyldidecylammonium, dibutyldiundecylammonium, dibutyldidodecylammonium, dibutylditridecylammonium, dibutylditetradecylammonium, dibutyldipentadecylammonium, dibutyldihexadecylammonium, dibutyldiheptadecylammonium, dibutyldioctadecylammonium and dibutyldinonadecylammonium salts where C2 is replaced with CHOH and C4 is replaced with O;

tributyldecylammonium, tributylundecylammonium, tributyldodecylammonium, tributyltridecylammonium, tributyltetradecylammonium, tributylpentadecylammonium, tributylhexadecylammonium, tributylheptadecylammonium, tributyloctadecylammonium, tributylnonadecylammonium, tripentyldecylammonium, tripentylundecylammonium, tripentyldodecylammonium, tripentyltridecylammonium, tripentyltetradecylammonium, tripentylpentadecylammonium, tripentylhexadecylammonium, tripentylheptadecylammonium, tripentyloctadecylammonium, tripentylnonadecylammonium, propyldibutyldecylammonium, propyldibutylundecylammonium, propyldibutyldodecylammonium, propyldibutyltridecylammonium, propyldibutyltetradecylammonium, propyldibutylpentadecylammonium, propyldibutylhexadecylammonium, propyldibutylheptadecylammonium, propyldibutyloctadecylammonium, propyldibutylnonadecylammonium, allyldibutyldecylammonium, allyldibutylundecylammonium, allyldibutyldodecylammonium, allyldibutyltridecylammonium, allyldibutyltetradecylammonium, allyldibutylpentadecylammonium, allyldibutylhexadecylammonium, allyldibutylheptadecylammonium, allyldibutyloctadecylammonium, allyldibutylnonadecylammonium, methallyldibutyldecylammonium, methallyIdibutylundecylammonium, methallyldibutyldodecylammonium, methallyldibutyltridecylammonium, methallyldibutyltetradecylammonium, methallyldibutylpentadecylammonium, methallyldibutylhexadecylammonium, methallyldibutylheptadecylammonium, methallyldibutyloctadecylammonium, methallyldibutylnonadecylammonium, dibutyldidecylammonium, dibutyldiundecylammonium, dibutyldidodecylammonium, dibutylditridecylammonium, dibutylditetradecylammonium, dibutyldipentadecylammonium, dibutyldihexadecylammonium, dibutyldiheptadecylammonium, dibutyldioctadecylammonium and dibutyldinonadecylammonium salts where C2 is replaced with CHCH₃, C3 is replaced with O and C4 is replaced with C═O;

tributyldecylammonium, tributylundecylammonium, tributyldodecylammonium, tributyltridecylammonium, tributyltetradecylammonium, tributylpentadecylammonium, tributylhexadecylammonium, tributylheptadecylammonium, tributyloctadecylammonium, tributylnonadecylammonium, tripentyldecylammonium, tripentylundecylammonium, tripentyldodecylammonium, tripentyltridecylammonium, tripentyltetradecylammonium, tripentylpentadecylammonium, tripentylhexadecylammonium, tripentylheptadecylammonium, tripentyloctadecylammonium, tripentylnonadecylammonium, propyldibutyldecylammonium, propyldibutylundecylammonium, propyldibutyldodecylammonium, propyldibutyltridecylammonium, propyldibutyltetradecylammonium, propyldibutylpentadecylammonium, propyldibutylhexadecylammonium, propyldibutylheptadecylammonium, propyldibutyloctadecylammonium, propyldibutylnonadecylammonium, allyldibutyldecylammonium, allyldibutylundecylammonium, allyldibutyldodecylammonium, allyldibutyltridecylammonium, allyldibutyltetradecylammonium, allyldibutylpentadecylammonium, allyldibutylhexadecylammonium, allyldibutylheptadecylammonium, allyldibutyl-octadecylammonium, allyldibutylnonadecylammonium, methallyldibutyldecylammonium, methallyldibutylundecylammonium, methallyldibutyldodecylammonium, methallyldibutyltridecylammonium, methallyldibutyltetradecylammonium, methallyldibutylpentadecylammonium, methallyldibutylhexadecylammonium, methallyldibutylheptadecylammonium, methallyldibutyloctadecylammonium, methallyldibutylnonadecylammonium, dibutyldidecylammonium, dibutyldiundecylammonium, dibutyldidodecylammonium, dibutylditridecylammonium, dibutylditetradecylammonium, dibutyldipentadecylammonium, dibutyldihexadecylammonium, dibutyldiheptadecylammonium, dibutyldioctadecylammonium and dibutyldinonadecylammonium salts where C3 is replaced with O and C4 is replaced with C═O;

tributyldecylammonium, tributylundecylammonium, tributyldodecylammonium, tributyltridecylammonium, tributyltetradecylammonium, tributylpentadecylammonium, tributylhexadecylammonium, tributylheptadecylammonium, tributyloctadecylammonium, tributylnonadecylammonium, tripentyldecylammonium, tripentylundecylammonium, tripentyldodecylammonium, tripentyltridecylammonium, tripentyltetradecylammonium, tripentylpentadecylammonium, tripentylhexadecylammonium, tripentylheptadecylammonium, tripentyloctadecylammonium, tripentylnonadecylammonium, propyldibutyldecylammonium, propyldibutylundecylammonium, propyldibutyldodecylammonium, propyldibutyltridecylammonium, propyldibutyltetradecylammonium, propyldibutylpentadecylammonium, propyldibutylhexadecylammonium, propyldibutylheptadecylammonium, propyldibutyloctadecylammonium, propyldibutylnonadecylammonium, allyldibutyldecylammonium, allyldibutylundecylammonium, allyldibutyldodecylammonium, allyldibutyltridecylammonium, allyldibutyltetradecylammonium, allyldibutylpentadecylammonium, allyldibutylhexadecylammonium, allyldibutylheptadecylammonium, allyldibutyloctadecylammonium, allyldibutylnonadecylammonium, methallyldibutyldecylammonium, methallyldibutylundecylammonium, methallyldibutyldodecylammonium, methallyldibutyltridecylammonium, methallyldibutyltetradecylammonium, methallyldibutylpentadecylammonium, methallyldibutylhexadecylammonium, methallyldibutylheptadecylammonium, methallyldibutyloctadecylammonium, methallyldibutylnonadecylammonium, dibutyldidecylammonium, dibutyldiundecylammonium, dibutyldidodecylammonium, dibutylditridecylammonium, dibutylditetradecylammonium, dibutyldipentadecylammonium, dibutyldihexadecylammonium, dibutyldiheptadecylammonium, dibutyldioctadecylammonium and dibutyldinonadecylammonium salts where C3 is replaced with O;

tributyldecylammonium, tributylundecylammonium, tributyldodecylammonium, tributyltridecylammonium, tributyltetradecylammonium, tributylpentadecylammonium, tributylhexadecylammonium, tributylheptadecylammonium, tributyloctadecylammonium, tributylnonadecylammonium, tripentyldecylammonium, tripentylundecylammonium, tripentyldodecylammonium, tripentyltridecylammonium, tripentyltetradecylammonium, tripentylpentadecylammonium, tripentylhexadecylammonium, tripentylheptadecylammonium, tripentyloctadecylammonium, tripentylnonadecylammonium, propyldibutyldecylammonium, propyldibutylundecylammonium, propyldibutyldodecylammonium, propyldibutyltridecylammonium, propyldibutyltetradecylammonium, propyldibutylpentadecylammonium, propyldibutylhexadecylammonium, propyldibutylheptadecylammonium, propyldibutyloctadecylammonium, propyldibutylnonadecylammonium, allyldibutyldecylammonium, allyldibutylundecylammonium, allyldibutyldodecylammonium, allyldibutyltridecylammonium, allyldibutyltetradecylammonium, allyldibutylpentadecylammonium, allyldibutyhexadecylammonium, allyldibutylheptadecylammonium, allyldibutyloctadecylammonium, allyldibutylnonadecylammonium, methallyldibutyldecylammonium, methallyldibutylundecylammonium, methallyldibutyldodecylammonium, methallyldibutyltridecylammonium, methallyldibutyltetradecylammonium, methallyldibutylpentadecylammonium, methallyldibutylhexadecylammonium, methallyldibutylheptadecylammonium, methallyldibutyloctadecylammonium, methallyldibutylnonadecylammonium, dibutyldidecylammonium, dibutyldiundecylammonium, dibutyldidodecylammonium, dibutylditridecylammonium, dibutylditetradecylammonium, dibutyldipentadecylammonium, dibutyldihexadecylammonium, dibutyldiheptadecylammonium, dibutyldioctadecylammonium and dibutyldinonadecylammonium salts where C3 is replaced with O and C5 is replaced with CHOH; and

tributyldecylammonium, tributylundecylammonium, tributyldodecylammonium, tributyltridecylammonium, tributyltetradecylammonium, tributylpentadecylammonium, tributylhexadecylammonium, tributylheptadecylammonium, tributyloctadecylammonium, tributylnonadecylammonium, tripentyldecylammonium, tripentylundecylammonium, tripentyldodecylammonium, tripentyltridecylammonium, tripentyltetradecylammonium, tripentylpentadecylammonium, tripentylhexadecylammonium, tripentylheptadecylammonium, tripentyloctadecylammonium, tripentylnonadecylammonium, propyldibutyldecylammonium, propyldibutylundecylammonium, propyldibutyldodecylammonium, propyldibutyltridecylammonium, propyldibutyltetradecylammonium, propyldibutylpentadecylammonium, propyldibutylhexadecylammonium, propyldibutylheptadecylammonium, propyldibutyloctadecylammonium, propyldibutylnonadecylammonium, allyldibutyldecylammonium, allyldibutylundecylammonium, allyldibutyldodecylammonium, allyldibutyltridecylammonium, allyldibutyltetradecylammonium, allyldibutylpentadecylammonium, allyldibutyhexadecylammonium, allyldibutylheptadecylammonium, allyldibutyloctadecylammonium, allyldibutylnonadecylammonium, methallyldibutyldecylammonium, methallyldibutylundecylammonium, methallyldibutyldodecylammonium, methallyldibutyltridecylammonium, methallyldibutyltetradecylammonium, methallyldibutylpentadecylammonium, methallyldibutylhexadecylammonium, methallyldibutylheptadecylammonium, methallyldibutyloctadecylammonium, methallyldibutylnonadecylammonium, dibutyldidecylammonium, dibutyldiundecylammonium, dibutyldidodecylammonium, dibutylditridecyl-ammonium, dibutylditetradecylammonium, dibutyldipentadecylammonium, dibutyldihexadecylammonium, dibutyldiheptadecylammonium, dibutyldioctadecylammonium and dibutyldinonadecylammonium salts where C9 and C10 are replaced with CH═CH.

Also suitable are phosphonium compounds corresponding to these ammonium compounds. Finally, mixtures of such onium compounds are suitable or in many cases preferred for use with the present invention. A number of other examples have been disclosed and described in U.S. Pat. Nos. 5,460,728 and 5,648,575 and such compounds can also be used with the present invention.

Suitable anionic compounds for use with these cationic LDHIs include, but are not necessarily limited to, alcohol sulfates that contain at least 4 carbon atoms; alcohol ether sulfates where the alcohol group contains at least 1 carbon atom and an ether linkage derived from at least one group consisting of ethylene oxide, propylene oxide, butylene oxide and styrene oxide; mono- or di-phosphate esters where the alcohol contains at least one carbon atom or contains an ether linkage derived from at least one group consisting of ethylene oxide, propylene oxide, butylene oxide and styrene oxide; sulfonic acids having at least 4 carbon atoms; phosphonic acids having at least 4 carbon atoms; carboxylic acids having at least 4 carbon atoms; taurates derived from a carboxylic acid having at least 1 carbon atom; and sarcosinates derived from carboxylic acids that contain at least 1 carbon atom; and forms of the anionic compounds as inorganic salts of the group consisting of lithium, sodium, potassium and ammonium; and organic salts with an amine having from 1 to 20 carbon atoms.

Non-ionic compounds suitable for use with these cationic LDHIs take in, but are not necessarily limited to, ethoxylated, propoxylated, and/or butoxylated alcohols, phenols, carboxylic acids and amines, sorbitan esters and ethoxylated, propoxylated, and/or butoxylated sorbitan esters; alkanolamine esters and/or amides.

Acceptable amphoteric compounds betaines derived from amines that contain at least 3 carbon atoms; alkyldimethyl-3-sulfopropylammonium inner salts; and alkyldimethyl-2-hydroxy-3-sulfopropylammonium inner salts. These descriptions of suitable anionic compounds, non-ionic compounds, and amphoteric compounds also apply to other types of ion pairs described herein. Suitable amphoteric compounds contain both cationic and anionic components of course; in this case the cationic component may be contributed from a source such as a strong acid. The anion may be Cl⁻, Br⁻, in essence any of the anions useful for the onium compounds discussed supra at formula A. As noted, sodium dodecyl sulfate and ammonium alkyl ether sulfates are two more specific counter-ions found to be effective when used with cationic LDHIs.

The operational active molar ratio range of first hydrate inhibitor component to second counter-ion component for this invention may be from about 100 to 1 to about 1 to 100. In another non-limiting embodiment, the range may be from about 100 to 10 to about 10 to 100. In an alternate non-restrictive embodiment, the range of the gas hydrate inhibiting ion to the counter ion may range from about 100 to 30 to about 30 to 100. In the context of this invention, molar ratios are close to weight ratios.

The contacting of the ion pair with the mixture of hydrocarbon, water and hydrate-forming guest molecules may be achieved by a number of ways or techniques, including, but not necessarily limited to, mixing, blending with mechanical mixing equipment or devices, stationary mixing setup or equipment, magnetic mixing or other suitable methods, other equipment and means known to one skilled in the art and combinations thereof to provide adequate contact and/or dispersion of the composition in the mixture. The contacting can be made in-line or offline or both. The various components of the composition may be mixed prior to or during contact, or both. As discussed, if needed or desired, the composition or some of its components may be optionally removed or separated mechanically, chemically, or by other methods known to one skilled in the art, or by a combination of these methods after the hydrate formation conditions are no longer present.

Because the present invention is particularly suitable for lower boiling hydrocarbons or hydrocarbon and/or non-hydrocarbon gases at ambient conditions with no more than five carbon atoms, the pressure of the condition is usually at or greater than atmospheric pressure (i.e. greater than or equal to about 101 kPa), preferably greater than about 1 MPa, and more preferably greater than about 5 MPa. The pressure in certain formations or processing plants or units could be much higher, say greater than about 20 MPa. There is no specific high pressure limit. The present method can be used at any pressure that allows formation of hydrocarbon gas hydrates.

The temperature of the condition for contacting is usually below, the same as, or not much higher than the ambient or room temperature. Lower temperatures tend to favor hydrate formation, thus requiring the treatment with the compositions of the present invention. At much higher temperatures, however, hydrocarbon hydrates may not form, thus obviating the need of carrying out any treatments.

It will be appreciated that it is very difficult, if not impossible, to predict in advance the proportions of ion pairs of this invention effective in inhibiting hydrocarbon hydrate formations in any given situation. There are a number of complex, interrelated factors that must be taken into account in determining the effective dosage or proportion, including, but not necessarily limited to, the proportion of water in the hydrocarbon, the nature of the hydrocarbon, the temperature and pressure conditions that the mixture of hydrocarbon and water are subject to, the particular ion pair hydrocarbon hydrate inhibitor employed, etc. Nevertheless, in the interest of attempting to provide some general guidance of effective proportions, relative to the water phase, the amount of the ion pair is less than 5 wt %, alternatively less than 2 wt %, and in another non-limiting embodiment is less than 1 wt %, but is limited only by what is economically feasible. In one non-limiting embodiment the lower limit is about 0.005 wt %, and alternatively is about 0.01 wt % and possibly is about 0.02 wt %. In a first non-limiting embodiment of the invention, the amount of ion pair may range from less than 5 wt % to 0.005 wt %, and in an alternate non-limiting embodiment may range from less than 2 wt % to about 0.02 wt %.

In addition to the ion pair of the invention, the hydrocarbon inhibitor composition may further comprise other additional components, including, but not limited to, different controlling chemistries such as corrosion inhibitors, wax inhibitors, scale inhibitors, asphaltene inhibitors and other hydrate inhibitors and/or solvents. Suitable solvents include, but are not limited to water; at least one oxygenated compound selected from C₁-C₆ alcohols, C₂-C₆ glycols, C₁-C₆ mono-aliphatic, preferably mono-alkyl, ethers of C₂-C₆ glycol, glycerin, C₁-C₆ mono-aliphatic, particularly mono-alkyl, ethers of glycerin, C₁-C₆ di-aliphatic, particularly dialkyl, ethers of glycerin, glycerin esters of C₁-C₆ carboxylate; tetrahydrofuran; N-methylpyrrolidone; sulfolane; C₃-C₁₀ ketones, and mixtures thereof. Examples of acceptable solvents in one non-limiting embodiment of the invention include water and liquid oxygenated materials such as methanol, ethanol, propanol, glycols like ethylene glycol, 1,2-propylene glycol, 1,3-propylene glycol, glycerin, esters and ethers of glycerin, CELLOSOLVE® (2-ethoxyethanol), CELLOSOLVE derivatives, 2-methoxyethanol, ethoxylated propylene glycols, ketones such as cyclohexanone and diisobutylketone, and mixtures thereof. The solvent is present in the total hydrocarbon hydrate inhibiting composition in the range of from 0 wt % to about 85 wt %, preferably from about 0 wt % to about 65 wt %, of the total composition, based on volume. CELLOSOLVE is a registered trademark of Union Carbide Corporation.

Because some of the ion pairs disclosed herein will be solids under ambient conditions, it is often preferred to use a suitable solvent as described above in the composition. This allows the formation of a homogeneous or uniform solution, suspension, emulsion or a combination of these, of all the components for easier mixing or distributing or dispersing the composition in the hydrocarbon/water fluid or system to be treated. As a result, more efficient and/or favorable contacting of the composition with the mixture comprising water and the hydrate-forming guest molecules can be effected.

The present invention also may be used in combination with other methods or processes, which have been known to one skilled in the art as discussed in the background to help inhibit formation of hydrates.

### EXPERIMENTAL SET-UP

All testing is isochoric. This results in the cell pressure dropping as the cell temperature is ramped or dropped from 72° F. to 40° F. (22° C. to 4° C.). The starting pressure is about 1500 psig (10.3 MPa), the final cell pressure at 40° F. (4° C.), before hydrate formation, varies, and is dependent on the test fluids (composition, liquid hydrocarbon ratio, etc.) employed. Generally, the cell pressure drops to the 1200 to 1300 psig range (8.3 to 9.0 MPa) before hydrate formation.

Testing is performed with a bank of modified sight flow indicators, which serve as pressure vessel reactors. Each reactor or cell is isolated from its companions, and is independently pressurized and contains its own, independent pressure transducer. Up to six reactors constitute a bank of test cells. A test is performed by immersing a bank of test cells in a common temperature controlled water bath.

Depending upon the experimental protocol, the water bath (and therefore the cells within) is gently rocked and/or held stationary at time intervals. Stationary intervals are designed to mimic pipeline shut-ins.

Other important procedural features include:
1. The bath water temperature and each pressure transducer are independently monitored and the data preserved by a computerized data acquisition system.
2. Each cell contains stainless steel ball(s) that provide agitation of the cell's contents when the water bath is rocked.
3. Often, one cell in every test bank is a control, containing either a reference inhibitor or none at all.
4. Tests employ either the shock cool method wherein the cells are placed in pre-chilled water or are ramp cooled from near room temperature to some target low temperature.
5. All cells are dissembled and meticulously cleaned with a proprietary system of solvents between each test.
6. Multiple repeats of a particular inhibitor blend are often made to provide a statistical sampling of a blend's performance.
7. Each cell has a window for visual observations.
8. Visual observations are made at irregular intervals to better ascertain the processes occurring within the cell and to confirm the results of the pressure data.

For the purpose of kinetic hydrate testing, the life and failure of a test blend is measured as the time expended before radical hydrate formation (retention time or time to failure). This point is denoted by a drop in pressure that is independent of a pressure drop due to a change in temperature.

In one non-limiting specific embodiment of the invention, sodium dodecylsulfate (SDS) is combined with the quaternary amine HI-M-PACT™ 4394 LDHI, having at least one appendage containing less than six carbon atoms and at least one appendage having more than six carbon atoms. The resulting ion pair has been shown to perform at active (dosage) levels equal to, if not less than the active (dosage) level of HI-M-PACT 4394 LDHI by itself. (The effective quaternary amine dosage is reduced from 0.59 wt % to 0.30 wt % active with the addition of as little as 0.04 wt % SDS, as tested with a Gulf of Mexico (GOM) condensate.) As noted, in some instances, the dosage level can be reduced to nearly half that normally or usually employed. This result reduces the amount of the quaternary amine required to control hydrates in the target matrix.

In a second non-limiting embodiment, an alcohol ether sulfate (AES) is added to HI-M-PACT 4394 LDHI with results parallel to those with SDS. The effective quaternary amine dosage is reduced from 0.75 wt % to 0.15 wt % with the addition of 0.12 wt % of the AES as tested with a GOM condensate.

Both the AES and SDS are known to have little or no independent hydrate inhibiting ability. (SDS is considered by some to be a hydrate promoter while AA testing with 0.30 wt % AES is known to fail with a GOM condensate.)

Another non-restrictive version of the invention dodecylbenzenesulfonic acid (DDBSA) is combined with the quaternary amine RE 4907. RE 4907 contains a small quaternary amine with appendages containing less than six carbon atoms. The resulting quaternary amine-DDBSA ion pair is shown to perform as an AA. Neither RE 4907 nor DDBSA demonstrate any applicable AA activity individually.

Many modifications may be made in the compositions and methods of this invention without departing from the spirit and scope thereof that are defined only in the appended claims. For example, the exact LDHI and counter-ions may be different from those explicitly mentioned herein. Various combinations of ion pairs other than those described here are also expected to find use in providing improved hydrate inhibitors. Further, combinations of ion pairs with mixtures of water, hydrocarbons and hydrate-forming guest molecules different from those exemplified herein would be expected to be successful within the context of this invention.

1. A method for inhibiting formation of hydrocarbon hydrates in a mixture comprising water and hydrate-forming guest molecules the method comprising contacting the mixture with an amount of an ion pair effective to inhibit formation of hydrocarbon hydrates, where the ion pair comprises:
a first component selected from the group consisting of cationic low dosage hydrate inhibitor (LDHI), anionic LDHI, amphoteric LDHI and non-ionic LDHI; and
a second counter-ion component, where in the case where the first component is:
a cationic LDHI, the second counter-ion component is selected from the group consisting of anionic compounds, non-ionic compounds and amphoteric compounds;
an anionic LDHI, the second counter-ion component is selected from the group consisting of non-ionic compounds, amphoteric compounds and cationic compounds;
an amphoteric LDHI or a non-ionic LDHI, the second counter-ion component is selected from the group consisting of anionic compounds, cationic compounds, non-ionic compounds and amphoteric compounds.

2. The method of claim 1 where the molar ratio of first component to second counter-ion component ranges from about 100 to 1 to about 1 to 100.

3. The method of claim 1 where the amount of the ion pair in the mixture ranges from about 0.005 to less than 5 wt % based on the water present.

4. A method for inhibiting formation of hydrocarbon hydrates in a mixture comprising water and hydrate-forming guest molecules the method comprising contacting the mixture with an amount of an ion pair effective to inhibit formation of hydrocarbon hydrates, where the ion pair comprises:
a cationic quaternary onium compound; and
a non-cationic counter-ion component selected from the group consisting of an anionic compound, a non-ionic compound and an amphoteric compound.

5. The method of claim 4 where the molar ratio of cationic component to non-cationic counter-ion component ranges from about 100 to 1 to about 1 to 100.

6. The method of claim 4 where the cationic quaternary onium compound has the formula: 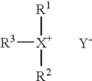
wherein
R¹ and R² each are independently selected from normal or branched alkyls containing a chain of at least 4 carbon atoms, having no, one or more substituents, or having no, one or more heteroatoms;
R³ is an organic moiety containing a chain of at least 4 carbon atoms, having no, one or more substituents, or having no, one or more heteroatoms;
X is S, N—R⁴ or P—R⁴;
R⁴, if present, is selected from H or an alkyl, aryl, alkylaryl, alkenylaryl or alkenyl group, preferably those having from about 1 to about 20 carbon atoms, having no, one or more substituents, or having no, one or more heteroatoms; and
Y⁻ is selected from the group consisting of hydroxide ion (OH⁻), halide ions such as Br⁻ and Cl⁻, carboxylate ions, such as benzoate (C₆H₅COO⁻), sulfate ion (SO₄⁼), organic sulfonate ions, such as 4-toluene sulfonate and CH₃SO₃⁻, and the like and mixtures thereof.

7. The method of claim 4 where the non-cationic counter-ion component is selected from the group consisting of
anionic compounds selected from the group consisting of alcohol sulfates that contain at least 4 carbon atoms; alcohol ether sulfates where the alcohol group contains at least 1 carbon atom and an ether linkage derived from at least one group consisting of ethylene oxide, propylene oxide, butylene oxide and styrene oxide; mono- or di-phosphate esters where the alcohol contains at least one carbon atom or contains an ether linkage derived from at least one group consisting of ethylene oxide, propylene oxide, butylene oxide and styrene oxide; sulfonic acids having at least 4 carbon atoms; phosphonic acids having at least 4 carbon atoms; carboxylic acids having at least 4 carbon atoms; taurates derived from a carboxylic acid having at least 1 carbon atom; and sarcosinates derived from carboxylic acids that contain at least 1 carbon atom; and acid forms of the anionic compounds as inorganic salts of the group consisting of lithium, sodium, potassium and ammonium; and organic salts with an amine having from 1 to 20 carbon atoms;
non-ionic compounds selected from the group consisting of ethoxylated, propoxylated, and/or butoxylated alcohols, phenols, carboxylic acids and amines; sorbitan esters and ethoxylated, propoxylated, and/or butoxylated sorbitan esters; alkanolamine esters and/or amides; and
amphoteric compounds selected from the group consisting of betaines derived from amines that contain at least 3 carbon atoms; alkyldimethyl-3-sulfopropylammonium inner salts; and alkyldimethyl-2-hydroxy-3-sulfopropylammonium inner salts.

8. The method of claim 4 where the non-cationic counter-ion component is selected from the group consisting of sodium dodecyl sulfate (SDS) and ammonium alkyl ether sulfate, and dodecylbenzenesulfonic acid (DDBSA).

9. The method of claim 4 where the amount of the ion pair in the mixture ranges from about 0.005 to less than 5 wt % based on the water present.

10. The method of claim 4 where the hydrate-forming guest molecule comprises at least one selected from the group consisting of methane, ethane, ethylene, acetylene, propane, propylene, methylacetylene, n-butane, isobutane, 1-butene, trans-2-butene, cis-2-butene, isobutene, butene mixtures, isopentane, pentenes, natural gas, carbon dioxide, hydrogen sulfide, nitrogen, oxygen, argon, krypton, xenon, and mixtures thereof.

11. A hydrocarbon mixture inhibited against hydrocarbon hydrate formation in the presence of water, the hydrocarbon mixture comprising
water;
hydrate-forming guest molecules; and
an ion pair in an amount effective to inhibit formation of hydrocarbon hydrates, where the ion pair comprises:
a first component selected from the group consisting of cationic low dosage hydrate inhibitor (LDHI), anionic LDHI, amphoteric LDHI and non-ionic LDHI; and
a second counter-ion component, where in the case where the first component is:
a cationic LDHI, the second counter-ion component is selected from the group consisting of anionic compounds, non-ionic compounds and amphoteric compounds;
an anionic LDHI, the second counter-ion component is selected from the group consisting of non-ionic compounds, amphoteric compounds and cationic compounds;
an amphoteric LDHI or a non-ionic LDHI, the second counter-ion component is selected from the group consisting of anionic compounds, cationic compounds, non-ionic compounds and amphoteric compounds.

12. The mixture of claim 11 where the molar ratio of first component to second counter-ion component ranges from about 100 to 1 to about 1 to 100.

13. The mixture of claim 11 where the amount of the ion pair in the mixture ranges from about 0.005 to less than 5 wt % based on the water present.

14. A hydrocarbon mixture inhibited against hydrocarbon hydrate formation in the presence of water, the hydrocarbon mixture comprising:
water;
hydrate-forming guest molecules; and
an ion pair in an amount effective to inhibit formation of hydrocarbon hydrates, where the ion pair comprises:
a cationic quaternary onium compound; and
a non-cationic counter-ion component selected from the group consisting of an anionic compound, a non-ionic compound and an amphoteric compound.

15. The mixture of claim 14 where the molar ratio of cationic component to non-cationic counter-ion component ranges from about 100 to 1 to about 1 to 100.

16. The mixture of claim 14 where the cationic quaternary onium compound has the formula: 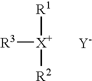
wherein
R¹ and R² each are independently selected from normal or branched alkyls containing a chain of at least 4 carbon atoms, having no, one or more substituents, or having no, one or more heteroatoms;
R³ is an organic moiety containing a chain of at least 4 carbon atoms, having no, one or more substituents, or having no, one or more heteroatoms;
X is S, N—R⁴ or P—R⁴;
R⁴, if present, is selected from H or an alkyl, aryl, alkylaryl, alkenylaryl or alkenyl group, preferably those having from about 1 to about 20 carbon atoms, having no, one or more substituents, or having no, one or more heteroatoms; and
Y⁻ is selected from the group consisting of hydroxide ion (OH⁻), halide ions such as Br⁻ and Cl⁻, carboxylate ions, such as benzoate (C₆H₅COO⁻), sulfate ion (SO₄⁼), organic sulfonate ions, such as 4-toluene sulfonate and CH₃SO₃⁻, and the like and mixtures thereof.

17. The mixture of claim 13 where the non-cationic counter-ion component is selected from the group consisting of
anionic compounds selected from the group consisting of alcohol sulfates that contain at least 4 carbon atoms; alcohol ether sulfates where the alcohol group contains at least 1 carbon atom and an ether linkage derived from at least one group consisting of ethylene oxide, propylene oxide, butylene oxide and styrene oxide; mono- or di-phosphate esters where the alcohol contains at least one carbon atom or contains an ether linkage derived from at least one group consisting of ethylene oxide, propylene oxide, butylene oxide and styrene oxide; sulfonic acids having at least 4 carbon atoms; phosphonic acids having at least 4 carbon atoms; carboxylic acids having at least 4 carbon atoms; taurates derived from a carboxylic acid having at least 1 carbon atom; and sarcosinates derived from carboxylic acids that contain at least 1 carbon atom; and acid forms of the anionic compounds as inorganic salts of the group consisting of lithium, sodium, potassium and ammonium; and organic salts with an amine having from 1 to 20 carbon atoms;
non-ionic compounds selected from the group consisting of ethoxylated, propoxylated, and/or butoxylated alcohols, phenols, carboxylic acids and amines; sorbitan esters and ethoxylated, propoxylated, and/or butoxylated sorbitan esters; alkanolamine esters and/or amides; and
amphoteric compounds selected from the group consisting of betaines derived from amines that contain at least 3 carbon atoms; alkyldimethyl-3-sulfopropylammonium inner salts; and alkyldimethyl-2-hydroxy-3-sulfopropylammonium inner salts.

18. The mixture of claim 14 where the non-cationic counter-ion component is selected from the group consisting of sodium dodecyl sulfate (SDS) and ammonium alkyl ether sulfate, and dodecylbenzenesulfonic acid (DDBSA).

19. The mixture of claim 14 where the amount of the ion pair in the mixture ranges from about 0.005 to less than 5 wt % based on the water present.

20. The mixture of claim 14 where the hydrate-forming guest molecule comprises one selected from the group consisting of methane, ethane, ethylene, acetylene, propane, propylene, methylacetylene, n-butane, isobutane, 1-butene, trans-2-butene, cis-2-butene, isobutene, butene mixtures, isopentane, pentenes, natural gas, carbon dioxide, hydrogen sulfide, nitrogen, oxygen, argon, krypton, xenon, and mixtures thereof.
